(19) United States
(12) Patent Application Publication
(10) Pub. No.: US 2021/0214799 A1
(43) Pub. Date: Jul. 15, 2021

(54) METHOD AND KIT FOR THE CLASSIFICATION OF THYROID NODULES

(71) Applicants: Onkos Diagnósticos Moleculares Ltda ME, Ribeirão Preto (BR); Fundação Pio XII, Barretos (BR)

(72) Inventors: Marcos Tadeu Dos Santos, Ribeirão Preto (BR); Ana Lígia Buzolin, Chapadão do Sul (BR); Rozany Dufloth, São Paulo (BR); Eduardo Caetano Albino Da Silva, Ribeirão Preto (BR); Ricardo Ribeiro Gama, Vista Alegre (BR); André Lopes, São Paulo (BR)

(73) Assignees: Onkos Diagnósticos Moleculares Ltda ME, Ribeirão Preto (BR); Fundação Pio XII, Barretos (BR)

(21) Appl. No.: 16/967,500

(22) PCT Filed: Feb. 22, 2019
(86) PCT No.: PCT/BR2019/050053

§ 371 Date: Aug. 5, 2020

(30) Foreign Application Priority Data
Feb. 23, 2018 (BR) 102018003587-8

(51) Int. Cl. C12Q 1/6886; C12Q 1/686

(52) U.S. Cl. CPC C12Q 1/6886; C12Q 2600/158; C12Q 2600/178; C12Q 1/686

(57) ABSTRACT

A method and kit for the classification of thyroid nodules that allows a more precise identification of the type of thyroid nodule as being malignant, benign or subclassifications, including at least one step of measuring the expression level of at least one normalizing microRNA and at least one discriminating microRNA and at least one step of correlation between the expression level of at least one normalizing microRNA and at least one discriminating microRNA.

### FIELD OF THE INVENTION

The present invention relates to a method and a kit for the classification of thyroid nodules. The present invention belongs to the fields of genetics and molecular biology.

### BACKGROUND OF THE INVENTION

Thyroid nodules, the main clinical manifestation of several thyroid diseases, are commonly observed in medical practice. Ultrasound studies in random populations show that up to 68% of the population may develop a thyroid nodule at some point in life.

International clinical guidelines from medical societies of endocrinology and metabolism, head and neck surgery and cytopathology recommend that all patients with nodule(s) greater than 1 cm and normal thyroid function should be punctured for cytological evaluation by Fine-Needle Aspiration Puncture (FNAP), preferably ultrasound-guided, which is a technique regarded as a “gold standard” also by the American Thyroid Association (ATA) for evaluating a nodule benignity or malignancy.

Cytological evaluation of these nodules (from the material collected by FNAP) shows that about 60 to 80% are benign lesions (“Bethesda II” Classification), in which the patient usually only observes and follows up the nodule, and usually does not require associated interventions or treatments. On the other hand, about 5 to 15% of FNAP biopsies on thyroid nodules are classified as malignant (“Bethesda VI” Classification). In these cases, the internationally adopted standard treatment is the total or partial removal of the thyroid gland, a situation that requires the patient to undergo a surgical procedure that may cause several problems, such as hypocalcemia, temporary or chronic hoarseness, infections and recurrent laryngeal nerve injuries. The most annoying consequence of this procedure is the need for hormone replacement for the rest of the patient's life.

The problem is that between 15 and 30% of the patients with punctured nodules have a result known as “indeterminate nodule” (“Bethesda III”, “Bethesda IV” or “Bethesda V” Classes), that is, the (cyto)pathologist who analyzed the punctured contents of the nodule does not have sufficient elements to discriminate the lesion between “benign” or “malignant”. In these cases, most international guidelines also recommend treatment through the surgical procedure for total or partial thyroid removal, since the risk of malignancy for indeterminate lesions is relevant, ranging from 5 to 75%.

However, the vast majority of indeterminate nodule thyroid surgeries are unnecessary, as about 70 to 80% of these nodules are identified as “benign” during postoperative histological analysis. It is also evident the huge unnecessary expenditure of the health system with this procedure. Not to mention the clinical complications that a surgery of this modality may unnecessarily bring to the patient: Thyroid surgeries have perioperative mortality rates between 0.1 and 0.2%. Serious or permanent non-lethal complications, including recurrent laryngeal nerve injuries, hypocalcemia, rebleeding, and infections, occur in 2 to 10% of the surgeries. In Brazil, 34.7% of thyroidectomies performed at a university hospital present some of these complications, including hypoparathyroidism in 8.8% of the cases. In addition to the risks inherent in the surgical procedure, levothyroxine replacement will be necessary throughout life. In Brazil only, there is an estimated of about 40,000 unnecessary thyroid surgeries per year.

These data confirm the critical need for improvements in preoperative diagnostic procedures for patients with thyroid nodules classified as indeterminate on the cytological test after FNAP.

Molecular techniques have been increasingly used in the evaluation of indeterminate thyroid nodules and new tests have appeared in the recent years with the purpose of helping to make the best clinical decision for these cases.

The analysis of gene mutations, such as BRAF, TERT, RAS, TP53 and various fusions in either single or panel analysis has shown good specificity and positive predictive value (PPV) and are commonly used as tests to identify malignancy (Rule-in tests), and not in order to avoid unnecessary surgeries.

On the other hand, molecular classifiers that work with RNA genetic signatures have proven to be great tools in identifying benign lesions (Rule-out tests), with the clear purpose of reducing unnecessary surgeries, as they have good results in sensitivity and negative predictive value (NPV).

A review and meta-analysis study published in 2018 by Vargas Salas et al. proposed specific cut-off points (thresholds) for the definition of molecular tests for indeterminate thyroid nodules. This study proposes that an optimal test should have a sensitivity of at least 92% and a specificity of at least 80% to have a clinical performance within the thyroid cancer prevalence commonly observed worldwide (20 to 40%) to achieve a NPV of at least 94% and a PPV of at least 60%.

Another very desired feature for this type of test is the ability to be performed from the FNAP material already collected from the patient, without the need to perform a new collection. The FNAP collection procedure is invasive, painful and stressful. In addition, analyzing exactly the same cells that were classified as “indeterminate” is a clear and desired technical advantage.

An important limitation of the currently available solutions is that there is no molecular test for indeterminate nodules that is performed from the sample already collected and that achieves the minimum clinical performance values proposed by Vargas-Salas et al. in 2018 considered both a rule-in and a rule-out test.

Currently available tests that are performed from material already collected are either rule-in or rule-out. Currently available tests that are considered rule-in and rule-out require a new FNAP from the patient.

Another important limitation is that currently available tests have a high financial cost and are performed in central laboratories primarily in the United States, which limits the access of patients to the use of these technologies worldwide and considerably delays the lead time.

The use of microRNAs (miRNAs—small non-encoding single-stranded RNAs of 18 to 25 nucleotides, participating in the process of regulating gene expression) as unique biomarkers or in identifying their expression profiles has been shown as one of the most promising technologies for the diagnosis, especially for cancer, since they effectively participate in the regulation of several processes of cell growth and differentiation.

Recently, new alternatives to molecular classifiers for indeterminate nodules that use microRNAs as their primary target have been commercially available and have shown an increase in specificity, reaching 72% and 85% when combined with panel analysis of specific mutations.

In the search for the state of the art in scientific and patent literatures, the following documents dealing with the subject matter were found:

The document WO2015175660A1, entitled “miRNA expression signature in the classification of thyroid tumors”, discloses methods for the classification of thyroid tumors using microRNA molecules associated with specific thyroid tumors.

The document WO2010129934A2, entitled “Methods and compositions for diagnosis of thyroid conditions”, discloses compositions, kits and methods for molecular profiles and cancer diagnosis, including cancer-associated genomic DNA markers. Said document discloses molecular profiles associated with thyroid cancer, methods for determining molecular profiles, and result analysis methods to provide a diagnosis.

The document WO2013066678A1, entitled “MicroRNA expression profiling of thyroid cancer”, discloses screening or diagnostic methods for thyroid cancer or a potential for developing thyroid cancer that include determining the expression levels of at least one miRNA selected from a specific group of miRNAs and compare the miRNA expression levels of the subject with a control subject who has no thyroid cancer or nodular hyperplasia.

The document WO2012068400A2, entitled “MiRNAs as biomarkers for distinguishing benign from malignant thyroid neoplasms”, discloses methods and compositions for identifying a miRNA profile for a particular condition, such as thyroid nodules or thyroid cancer, and using the profile in diagnosing a patient for a condition such as thyroid nodules or thyroid cancer.

Thus, from what is clear from the researched literature, no documents were found anticipating or suggesting the teachings of the present invention, so that the solution proposed herein, in the inventors point of view, has novelty and inventive activity compared to the state of the art.

The invention is disclosed as an alternative to solve the various problems and drawbacks found in the existing indeterminate thyroid nodules classification methods intended to move towards an ideal method by solving the problems of specificity, sensitivity, accessibility, convenience and cost for the classification of indeterminate thyroid nodules.

### SUMMARY OF THE INVENTION

The present invention is intended to solve the frequent prior art problems by means of an improved method for the classification of thyroid nodules. The method of the invention comprises at least one step of measuring the expression level of microRNAs and at least one step of correlation between the level of normalizing microRNA expression and at least one discriminating microRNA; wherein said normalizing microRNA is selected in combinations of one to six from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA, and

wherein said discriminating microRNA is selected from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA or combinations thereof.

In a second aspect, the present invention defines a kit for the classification of thyroid nodules, comprising:
materials for measuring the expression level of at least one normalizing microRNA and at least one discriminating microRNA; and
at least one means for correlating the expression level of one to six normalizing microRNA and at least one discriminating microRNA;

wherein said normalizing microRNA is as described above, and wherein said discriminating microRNA is as described above.

The inventive concept common to all claimed protection contexts is the disclosed solution to the problem of a more accurate classification of thyroid nodules, which includes one or more of the normalizing miRNAs and one or more of the discriminating miRNAs and/or a specific form of correlation therebetween.

These and other objects of the invention will be immediately appreciated by those skilled in the art and companies having an interest in the segment, and will be described in sufficient details for their reproduction in the following description.

### BRIEF DESCRIPTION OF THE DRAWINGS

In order to better define and clarify the contents of this patent application, the following figures are presented:

FIG. 1 Algorithm for the management of patients with thyroid nodules. The algorithm above was adapted and summarized from the Brazilian and American guidelines and consensus. See the highlight in red for the high number of unnecessary surgeries that are performed on patients with indeterminate nodules, evidencing problems in preoperative diagnostic procedures. SB=Bethesda System—Categories.

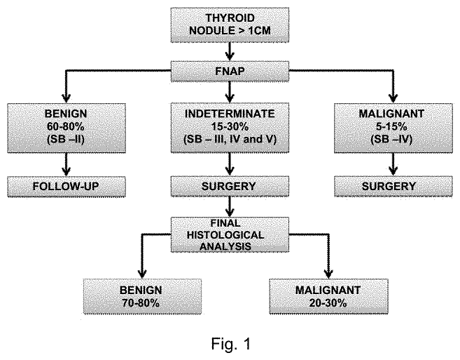

FIG. 2 shows a summarized flowchart of biomarkers selection, wherein 1—96 microRNAs selected based on the literature review; 2—Analysis of expression in 78 developmental samples (being 39 malignant, 39 benign); 3-65 microRNAs with expression in at least 95% of the samples; 4—10 microRNAs candidates for NORMALIZERS; 5—Generation of 175 normalizing values (N) (All possible combinations by the mean); 6—55 microRNAs candidates for DISCRIMINATORS (D); 7—Generation of 9625 (175×55) features (Each normalized discriminator for each normalizing value=2\hat{ }(N−D)), 8—Selection of the top-10 features using filter-based metaheuristic methods; 9—17 microRNAs (8 normalizing+9 discriminating) that make up the top-10 features; 10—Generation of classification algorithms based on Decision Tree Forest Machine-Learning techniques; 11—Training and testing (10-fold cross validation); 12—VALIDATION (expression analysis in the 95 validation samples (being 37 malignant, 58 benign)); 13—BLIND TESTING and 14—Best Algorithm: —25 features made up by microRNAs: 11 (6 normalizing and 5 discriminating).

FIG. 3 shows a detailed flowchart of the development and validation of microRNA selection, wherein 1-1205 patients with FNAP results available (January/2013-July/2016); 2-272 Patients with indeterminate Bethesda III, IV or V result in FNAP; 3-212 Patients with >2 FNAP slides and the corresponding postoperative tissue available; 4—Review by two independent pathologists (FNAP slides and postoperative tissue); 5-192 Patients eligible for the study; 6-40 Patients—postoperative tissue of BENIGN thyroid nodules that had been classified as indeterminate (Bethesda III, IV or V) in FNAP; 7-40 Patients—postoperative tissue of MALIGNANT thyroid nodules that had been classified as indeterminate (Bethesda III, IV or V) in FNAP; 8—RNA extraction; 9—Preamplification; 10—cDNA; 11—Real-Time PCR (TLDA Array Cards); 12—Expression data from 39 BENIGN samples; 13—Expression data from 39 MALIGNANT samples; 14—Selection of biomarkers; 15—Generation and selection of features; 16—Training and Testing (10-fold cross-validation); 17-70 Patients—FNAP slide of thyroid nodules classified as indeterminate (Bethesda III, IV or V) BENIGN; 18 —42 Patients—FNAP slide of thyroid nodules classified as indeterminate (Bethesda III, IV or V) MALIGNANT; 19—RNA extraction; 20—Preamplification; 21—cDNA; 22-Real-Time PCR (individual assays); 23—Expression data from 58 BENIGN samples; 24—Expression data from 37 MALIGNANT samples; 25—Generation of the same features predefined in the development; 26—BLIND TESTING and 27—FINAL ALGORITHM (Model with 11 microRNAs).

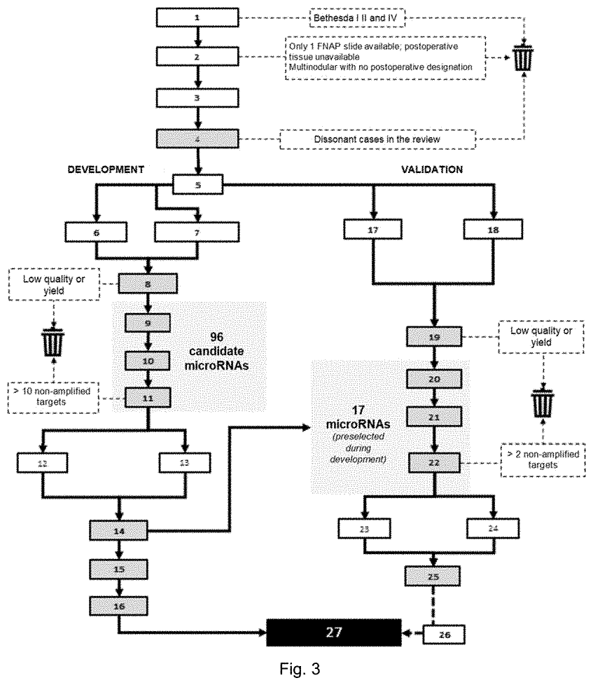

### DETAILED DESCRIPTION OF THE INVENTION

In a first object, the present invention defines a method for the classification of thyroid nodules comprising at least one step of measuring the expression level of microRNAs and at least one step of correlation between the level of normalizing microRNA expression and at least one discriminating microRNA; wherein said normalizing microRNA is selected in combinations of one to six from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA, and

wherein said discriminating microRNA is selected from the group consisting of dme-miR-7, hsa-let-7a, hsa-let-7b, hsa-let-7e, hsa-let-7f, hsa-let7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA or combinations thereof.

In one embodiment, said normalizing microRNA is selected from the group consisting of RNU48, hsa-miR-197, hsa-let-7b, hsa-miR-125a-5p, hsa-miR-103, hsa-let-7a, hsa-let-7e, hsa-miR-145 or combinations thereof.

In one embodiment, said discriminating microRNA is selected from the group consisting of hsa-miR-204, hsa-miR-152, hsa-miR-222, hsa-miR-181b, hsa-miR-146b, hsa-miR-155, hsa-miR-181a, hsa-miR-200b, hsa-miR-221 or combinations thereof.

In one embodiment, the normalizing microRNAs and the discriminating microRNAs are correlated from one or more of the following features:

TABLE 1

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |
| U | hsa-miR-145 | hsa-miR-222 |
| | hsa-miR-103 | |
| | hsa-miR-197 | |
| V | hsa-let-7e | hsa-miR-222 |
| | hsa-miR-197 | |
| W | hsa-miR-145 | hsa-miR-181a |
| | hsa-miR-197 | |
| Y | hsa-miR-145 | hsa-miR-221 |
| | hsa-miR-197 | |
| X | hsa-let-7A | hsa-miR-204 |
| | hsa-miR-103 | |
| | hsa-miR-125a-5p | |

In one embodiment, the normalizing microRNAs and the discriminating microRNAs are correlated from one or more of the following groups:

TABLE 2

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |

In one embodiment, the thyroid nodule classification consists of at least one of: benign, malignant, medullary thyroid cancer, papillary thyroid carcinoma and its variants, follicular thyroid carcinoma and its variants, insular thyroid carcinoma, noninvasive follicular neoplasm with papillary-like nuclear features (NIFTP), goiter and its variants, thyroiditis and its variants, adenomas and their variants, Hurthle cells thyroid and their variants and thyroid hyperplasias and their variants.

In one embodiment, when the classification is for medullary thyroid cancer, said discriminating microRNA is at least hsa-miR-375.

In one embodiment, the method comprises the steps of:

a) collecting thyroid tissue sample;
b) extracting nucleic acids from the sample of step (a);
c) measuring the expression level of at least one normalizing microRNA and at least one discriminating microRNA; and
d) correlating the data obtained in step (c) of the expression level of at least one normalizing microRNA and at least one discriminating microRNA.

In one or more embodiments, step (a) is performed by fine-needle aspiration puncture or biopsy; and/or step (c) is performed using a technique selected from the group consisting of RT-PCR, sequencing, microarrays, fragment analysis, gel electrophoresis, mass spectrometry or combinations thereof; and/or step (d) is performed by using an algorithm.

The method of the present invention works with either “fresh and liquid” samples of a new FNAP or with material extracted from previously prepared and stained cytology slides and coverslip.

In one embodiment, said algorithm uses single and/or committee decision trees system (RandonForest, ExtraTrees, C4.5, DecisionJungle, Boosted DecisionTrees, and others) to classify samples by analyzing the features generated by the joint normalization of the discriminating microRNAs by the normalizing ones.

In one embodiment, the method additionally comprises the steps of:

a1) preparing the sample collected in step (a) before performing step (b);
b1) purifying the nucleic acids obtained in step (b);
b2) cDNA synthesis from the nucleic acids obtained in step (b1), and optionally,
b3) preamplification prior to step (c).

In a second aspect, the present invention provides a kit for the classification of thyroid nodules, said kit comprising:
materials for measuring the expression level of at least one normalizing microRNA and at least one discriminating microRNA; and
at least one means for correlating the expression level of one to six normalizing microRNA and at least one discriminating microRNA;
   wherein said normalizing microRNA is selected from the group consisting of dme-miR-7, hsa-let-7a, hsa-let-7b, hsa-let-7e, hsa-let-7f, hsa-let7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA or combinations thereof, and
   wherein said discriminating microRNA is selected from the group consisting of dme-miR-7, hsa-let-7a, hsa-let-7b, hsa-let-7e, hsa-let-7f, hsa-let7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA or combinations thereof.

In one embodiment of the kit, said normalizing microRNA is selected from the group consisting of RNU48, hsa-miR-197, hsa-let-7b, hsa-miR-125a-5p, hsa-miR-103, hsa-let-7a, hsa-let-7e, hsa-miR-145 or combinations thereof, and wherein said discriminating microRNA is selected from the group consisting of hsa-miR-204, hsa-miR-152, hsa-miR-222, hsa-miR-181b, hsa-miR-146b, hsa-miR-155, hsa-miR-181a, hsa-miR-200b, hsa-miR-221 or combinations thereof.

In one embodiment, the kit additionally comprises:
material for collecting thyroid tissue sample or for extracting material from existing FNAP cytology slides;
material for preparing said sample;
reagents for extracting nucleic acids;
reagents for cDNA synthesis;
optionally, reagents for preamplification; and
material for measuring the expression level of microRNAs.

The method and kit of the present invention provide a more accurate classification of the tumor type of the thyroid nodule, being malignant or benign. In addition, the method of the present invention enables the use of either “fresh and liquid” sample from a new FNAP or with material extracted from previously prepared and stained cytology slides and coverslip, which provides a significant additional advantage over other existing methods.

### Examples—Embodiments

The examples shown herein are intended solely to exemplify one of the several ways of carrying out the invention, but without limiting the scope thereof.

### Example 1—Methodology for Measuring Expression Levels

### Remove Coverslip

Prior to beginning, reinforce the marking of the pathologist-delimited region with a strong permanent pen (Sharpie or similar) on the underside of the slide.

Prepare 4 Troughs as follows:

In a trough add xylol

In the second trough, add water.

In the third trough, 100% ethanol

In the fourth trough, add 70% ethanol

A) Method 1:

Freeze the slides in the freezer at −20° C.

Remove the slide from the freezer and remove the coverslip

Inside a fume hood, place the slides (without the coverslips) on the specific rack (“cradle”) and soak them in the xylol trough

Next, transfer the slides to the trough containing water.

Remove slides from water and dry.

B) Method 2:

Place the slides in the specific xylol rack

After stripping the coverslip, hydrate the slide in the following order:

Soak in the trough with xylol;

Transfer to the 100% ethanol trough

Transfer to the 70% ethanol trough

Transfer to the water trough

Dry

Collect the Cells from the Slide (Scraping)

For each sample, prepare one Trizol Reagent tube

After removing the coverslip by Method 1 or 2, scrape the marked area and transfer the scraped material to the respective tube.

Freeze the sample at −80° C.

Extraction Following Trizol Reagent Protocol:

Prepare 75% ethanol

Prepare an aliquot of chloroform;

Prepare an aliquot of isopropanol;

Thaw the homogenized sample in Trizol

Inside the fume hood, add chloroform to the sample;

Centrifuge

Collect the upper aqueous phase

Add isopropanol

Incubate at −80° C.

Centrifuge

Remove the supernatant

Add 75% ethanol

Centrifuge

Discard the supernatant

Repeat the ethanol wash

Dry

Add RNAse-free water

Resuspend the RNAse-free water pellet;

Quantify;

### RT-PCR

### RT-PCR Reaction Preparation

In pre-PCR laminar flow, prepare the mix for reverse transcription using TaqMan microRNA Reverse Transcription Kit reagents and the previously prepared RT pool

After preparing the mix, distribute it on the microplate.

Bring the plate to the workbench and add the sample RNA.

Place it in the thermal cycler with the following program:

TABLE 3

| Step | Time | Temperature |
|---|---|---|
| Hold | 30 min | 16° C. |
| Hold | 30 min | 42° C. |
| Hold | 5 min | 85° C. |
| Hold | ∞ | 4° C. |

### Preamplification (Optional Step)

### Preparing the Preamplification Reaction

Prepare the preamplification mix by following the volumes showed below:

TABLE 4

| REAGENT | 1X (uL) | 10X (uL) |
|---|---|---|
| Taqman Preamp MasterMix 2X | 6.25 | 62.5 |
| Preamp Primer Pool | 1.875 | 18.75 |
| H2O | 3.125 | 31.25 |
| subtotal | 11.25 | 112.5 |

After preparing the mix, distribute it on microplate microwells.

Bring the plate to the workbench and add 1.25 of the RT product.

Place it in the thermal cycler with the following cycling:

TABLE 5

| Step | Time | Temperature |
|---|---|---|
| Hold | 10 min | 95° C. |
| Hold | 2 min | 55° C. |
| Hold | 2 min | 72° C. |
| 12-17 Cycles | 15 sec | 95° C. |
| | 4 min | 60° C. |
| Hold | 10 min | 99.9° C. |
| Hold | ∞ | 4° C. |

Add H20-free RNAse to each microwell

Real Time PCR

Preparing the Real Time Plate with the Assays

Add each assay into the corresponding wells of a plate.

### Real Time Reaction

Prepare the reaction mix with the following volumes:

TABLE 6

| REAGENT | 1X (uL) | 28X (uL) |
|---|---|---|
| H2O | 1.0 | 28.0 |
| MasterMix | 5.0 | 140 |
| Preamp Product | 2.0 | 56 |
| subtotal | 8.0 | 224 |

Distribute the mix into each microwell of the plate containing previously pipetted assays.

Seal the plate

Real Time

Define cycling as per the table below:

TABLE 7

| Step | Time | Temperature |
|---|---|---|
| Hold | 10 min | 95° C. |
| 50 Cycles | 15 sec | 95° C. |
| | 15 sec | 60° C. |

At the end of the run, export the results to an Excel worksheet.

### Example 2—Selection of Biomarkers

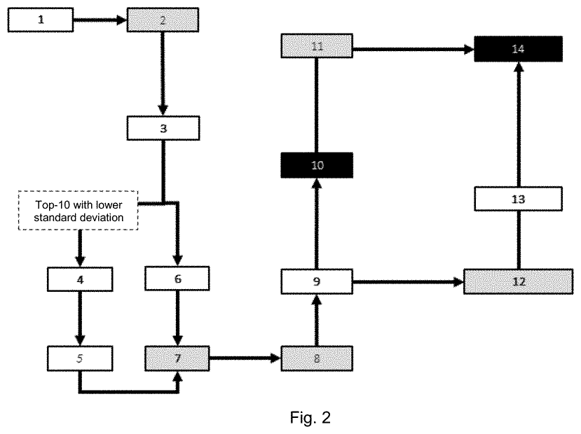

A summary of biomarkers selection can be seen in FIG. 2.

An initial list of 96 microRNAs for use in the method of the present invention was considered:

TABLE 8

| | | |
|---|---|---|
| dme-miR-7 | hsa-miR-15a | hsa-miR-30a-3p |
| hsa-let-7a | hsa-miR-16 | hsa-miR-30a-5p |
| hsa-let-7b | hsa-miR-17 | hsa-miR-30c-2* |
| hsa-let-7e | hsa-miR-181a | hsa-miR-30e-3p |
| hsa-let-7f | hsa-miR-181b | hsa-miR-31 |
| hsa-let-7g | hsa-miR-183 | hsa-miR-3151 |
| hsa-miR-1 | hsa-miR-18a | hsa-miR-346 |
| hsa-miR-101 | hsa-miR-18b | hsa-miR-34a |
| hsa-miR-103 | hsa-miR-190 | hsa-miR-34c |
| hsa-miR-106a | hsa-miR-191 | hsa-miR-365 |
| hsa-miR-106b | hsa-miR-195 | hsa-miR-375 |
| hsa-miR-10^{a} | hsa-miR-197 | hsa-miR-424 |
| hsa-miR-1179 | hsa-miR-199a-3p | hsa-miR-425-5p |
| hsa-miR-122 | hsa-miR-199b | hsa-miR-449b |
| hsa-miR-125a-3p | hsa-miR-200a | hsa-miR-503 |
| hsa-miR-125a-5p | hsa-miR-200b | hsa-miR-520b |
| hsa-miR-125b | hsa-miR-200c | hsa-miR-608 |
| hsa-miR-126 | hsa-miR-203 | hsa-miR-613 |
| hsa-miR-130b | hsa-miR-204 | hsa-miR-618 |
| hsa-miR-133^{a} | hsa-miR-205 | hsa-miR-642 |
| hsa-miR-136* | hsa-miR-208 | hsa-miR-651 |
| hsa-miR-136 | hsa-miR-208b | hsa-miR-7-2* |
| hsa-miR-138 | hsa-miR-20a | hsa-miR-885-5p |
| hsa-miR-144 | hsa-miR-20b | hsa-miR-9 |
| hsa-miR-145 | hsa-miR-21 | hsa-miR-933 |
| hsa-miR-146a | hsa-miR-221 | hsa-miR-99a |
| hsa-miR-146b | hsa-miR-222 | mmu-miR-137 |
| hsa-miR-149 | hsa-miR-23b | mmu-miR-187 |
| hsa-miR-150 | hsa-miR-26a | mmu-miR-451 |
| hsa-miR-151-5P | hsa-miR-26b | RNU44 |
| hsa-miR-152 | hsa-miR-29a | RNU48 |
| hsa-miR-155 | hsa-miR-302c | U6 snRNA |

This initial list of microRNAs was selected based on two criteria: 1) broad literature review (161 articles), seeking to identify studies that analyzed thyroid microRNAs and identified significant changes in their expression, and 2) bioinformatics analyses of samples available from public online databases (such as ArrayExpress). In this phase there was no differentiation between Discriminators and Normalizers.

The expression of these 96 initial candidate microRNAs in 80 indeterminate thyroid nodules (40 benign and 40 malignant—postoperative tissues) was analyzed and then only those microRNAs that expressed in at least 95% of the samples were selected. This analysis selected 65 microRNAs, being 10 candidates for normalizers and the remaining 55 candidates for discriminators. The selection of the 10 candidates for normalizers was carried out by analyzing the standard deviation. The 10 microRNAs with expression values (Ct) having a smaller standard deviation among all samples (benign and malignant) were preselected.

TABLE 9
Candidates for discriminators

| | |
|---|---|
| hsa-let-7f | hsa-miR-200a |
| hsa-let-7g | hsa-miR-200b |
| hsa-miR-106a | hsa-miR-200c |
| hsa-miR-106b | hsa-miR-203 |
| hsa-miR-10a | hsa-miR-204 |
| hsa-miR-125a-3p | hsa-miR-20a |
| hsa-miR-125b | hsa-miR-20b |
| hsa-miR-126 | hsa-miR-21 |
| hsa-miR-130b | hsa-miR-221 |
| hsa-miR-133a | hsa-miR-222 |
| hsa-miR-138 | hsa-miR-23b |
| hsa-miR-146a | hsa-miR-26a |
| hsa-miR-146b | hsa-miR-26b |
| hsa-miR-149 | hsa-miR-29a |
| hsa-miR-150 | hsa-miR-30a-3p |
| hsa-miR-151-5P | hsa-miR-30a-5p |
| hsa-miR-152 | hsa-miR-30c-2* |
| hsa-miR-155 | hsa-miR-30e-3p |
| hsa-miR-15a | hsa-miR-31 |
| hsa-miR-16 | hsa-miR-346 |
| hsa-miR-17 | hsa-miR-34a |
| hsa-miR-181a | hsa-miR-365 |
| hsa-miR-181b | hsa-miR-375 |
| hsa-miR-183 | hsa-miR-425-5p |
| hsa-miR-18a | hsa-miR-99a |
| hsa-miR-18b | mmu-miR-451 |
| hsa-miR-195 | RNU44 |
| hsa-miR-199a-3p | |

The selection of the 10 candidates for normalizers was carried out by analyzing the standard deviation. The 10 microRNAs with expression values (Ct) having a smaller standard deviation among all samples (benign and malignant) were preselected.

TABLE 10

Candidates for normalizers
hsa-let-7a
hsa-let-7b
hsa-let-7e
hsa-miR-103
hsa-miR-125a-5p
hsa-miR-145
hsa-miR-191
hsa-miR-197
RNU48
U6 snRNA

### Example 3—Generation of Normalizing Values

### Step 1

To generate unique values that will be used as normalizers, we made all possible combinations among the 10 candidates for normalizers, so that the final value was the average between the values.

### EXAMPLES

TABLE 11

| | |
|---|---|
| One microRNA: | the raw value of each candidate is used, therefore |
| | 10 single values |
| Two microRNAs: | all possible combinations of 2 microRNAs are |
| | made, the mean value is determined, therefore |
| | 45 possible combinations, 45 single |
| Three microRNAs: | all possible combinations of 3 microRNAs are |
| | made, therefore 36 possible combinations, |
| | 36 single values |
| Four microRNAs: | all possible combinations of 4 microRNAs are |
| | made, therefore 28 possible combinations, |
| . . . | 28 single values |
| Ten microRNAs: | all possible combinations of 10 microRNAs are |
| | made, therefore 1 single combination (mean of |
| | all 10), 1 single value |

At the end, all possible combinations come to a total of 175 combinations, therefore 175 single values to be used as normalization values.

### Step 2

Each of the 55 candidates for discriminator (D) is normalized by each of the 175 normalization values (N) generated above.

Normalization formula=2\hat{ }(N−D)

Therefore, each discriminator generates 175 normalized values. Each of these values is called a feature.

A feature is a value used in machine-learning to separate classes. The concept used in the invention was to identify a set of features that have distinct values between benign and malignant.

Therefore, 55 (discriminators)×175 (normalizing values)=9625 features are generated.

### Step 3

Filter-based metaheuristic methods were used to know which features best separate the benign and malignant classes. Examples include: Pearson's correlation, Mutual information score, Kendall's correlation coefficient, Spearman's correlation coefficient, Chi-squared statistic, Fisher score and Count based feature selection.

The results allowed us to observe which microRNAs make up the best features, to then analyze a smaller panel of microRNAs in new samples (validation samples).

### Validation

After applying the described selection methods, the present inventors observed that the top-10 features (i.e., with the highest discriminating power between classes) were made of these 17 microRNAs. We then analyzed the expression of these microRNAs in a new set of patients (other than those previously used) of 95 samples (validation samples), being 37 malignant and 58 benign, this time directly on the FNAP cytology slides.

### Example 4—Selection of Features

The features selected were:

TABLE 12

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |
| U | hsa-miR-145 | hsa-miR-222 |
| | hsa-miR-103 | |
| | hsa-miR-197 | |
| V | hsa-let-7e | hsa-miR-222 |
| | hsa-miR-197 | |
| W | hsa-miR-145 | hsa-miR-181a |
| | hsa-miR-197 | |
| Y | hsa-miR-145 | hsa-miR-221 |
| | hsa-miR-197 | |
| X | hsa-let-7A | hsa-miR-204 |
| | hsa-miR-103 | |
| | hsa-miR-125a-5p | |

Features A, B, C, D, E, F, G, G, I, J, K, L M, N, O, P, Q R, S and T described below provided the best results:

TABLE 13

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |
| | hsa-miR-103 | |
| | hsa-miR-125a-5p | |

### Example 5—Comparative Results

The results are showed in the following table.

TABLE 14

| | Algorithm # | 1 | 2 | 3 | 4 | 5 | 6 | 7 |
|---|---|---|---|---|---|---|---|---|
| | Features | A, B, C, | A, C, D, | B, C, D, | A, D, | B, C, D, | B, C, D, | A, B, |
| | | D, E | E, X | E, X | W, X | E, V | E, U | C, D |
| | # microRNAs | 11 | 11 | 11 | 11 | 13 | 12 | 10 |
| Development | Sensitivity | 90% | 82% | 87% | 84.60% | 84.60% | 84.60% | 79.50% |
| (training | Specificity | 93% | 56% | 64% | 69% | 82% | 72% | 74.40% |
| & | VPN | 91.50% | 76% | 83% | 81.80% | 84% | 82.40% | 78.40% |
| testing) | VPP | 93% | 65% | 71% | 73.30% | 82.50% | 75% | 75.60% |
| Validation | Sensitivity | 94.60% | 89.00% | 86.50% | 86.50% | 81% | 92% | 89% |
| | Specificity | 81% | 56.00% | 60% | 72% | 70% | 65% | 63% |
| | VPN | 96% | 89% | 87% | 89% | 85% | 92.50% | 90% |
| | VPP | 86% | 57% | 58% | 66.70% | 64% | 63% | 61% |

### Example 6—Algorithm

In the present invention various algorithms were also developed and selected until the most suitable one was chosen. This algorithm was developed, trained and tested by cross-validation (from 3 to 12 folds) with the features generated by the analysis of developmental samples. Next, the test of this algorithm was validated with the validation samples. Said algorithm uses single and/or committee decision trees system (1 to 100,000 trees) using RandonForest, ExtraTrees, C4.5, DecisionJungle, Boosted DecisionTrees, and others techniques, separately or as ensemble, to classify samples by analyzing the features generated by the joint normalization of the discriminating microRNAs by the normalizing ones.

### Example 7—Evaluation of the Use of microRNA Hsa-miR-375 as a Biomarker of Medullary Thyroid Carcinoma

In this embodiment, medullary thyroid carcinoma (MTC), which represents about 5-10% of the primary thyroid tumors and may behave more aggressively than well-differentiated thyroid tumors, has been evaluated, in addition to presenting high incidence of metastases. The identification of MTC at diagnosis in thyroid nodules may be extremely relevant for the definition of the correct surgical procedure to be performed, as well as suggesting investigation for other tumors and family MEN type-2 syndrome.

In this embodiment, the expression of hsa-mir-375 and 8 other normalizing microRNAs (including hsa-miR-103) was evaluated in 157 thyroid samples, being 42 from MTC, 77 benign and 38 MTC-non-malignant. Among these samples, 77 are from patients with indeterminate nodules and the analysis was performed by qPCR from cells extracted from FNAP cytology slides. Another 80 samples were obtained from the ArrayExpress public database (E-GEOD-40807) from postoperative tissue microarrays analysis. The discriminating potential of hsa-mir-375 was assessed by its fold-change relative to the normalizers candidates microRNAs.

The results show that analysis of hsa-mir-375 expression against normalizing microRNAs showed that when fold-change is greater than or equal to 3.0, or greater than or equal to 2.5, this relationship has the potential to discriminate “MTC” vs “benign or MTC-non-malignant” samples with 92% specificity, 78.6% sensitivity, 92% positive predictive value, 78.6% negative predictive value and 88.4% accuracy.

The results allow us to conclude that hsa-mir-375 has a high potential to be used as a biomarker for MTC at diagnosis, including the analysis of its expression by qPCR in indeterminate thyroid nodules from cells fixed in FNAP cytology slides, thus being able to objectively assist in medical decision-making about the best surgical approach and investigation to be performed.

Those skilled in the art will enhance the knowledge presented herein and may practice the invention in the embodiments disclosed and in other variants within the scope of the appended claims.

1. A method for classifying thyroid nodule comprising at least one step of measuring the expression level of microRNAs and at least one step of correlation between the expression level of normalizing microRNA with at least one discriminating microRNA;
wherein said normalizing microRNA is selected in combinations of one to six from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, and U6 snRNA; and
wherein said discriminating microRNA is selected from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA, and combinations thereof.

2. The method according to claim 1, wherein said normalizing microRNA is selected from the group consisting of RNU48, hsa-miR-197, hsa-let-7b, hsa-miR-125a-5p, hsa-miR-103, hsa-let-7a, hsa-let-7e, and hsa-miR-145, and/or said discriminating microRNA is selected from the group consisting of hsa-miR-204, hsa-miR-152, hsa-miR-222, hsa-miR-181b, hsa-miR-146b, hsa-miR-155, hsa-miR-181a, hsa-miR-200b, hsa-miR-221, and combinations thereof.

3. The method according to claim 2, wherein the normalizing microRNAs and the discriminating microRNAs are correlated from one or more of the following features:

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |
| U | hsa-miR-145 | hsa-miR-222 |
| | hsa-miR-103 | |
| | hsa-miR-197 | |
| V | hsa-let-7e | hsa-miR-222 |
| | hsa-miR-197 | |
| W | hsa-miR-145 | hsa-miR-181a |
| | hsa-miR-197 | |
| Y | hsa-miR-145 | hsa-miR-221 |
| | hsa-miR-197 | |
| X | hsa-let-7A | hsa-miR-204 |
| | hsa-miR-103 | |
| | hsa-miR-125a-5p | |

4. The method according to claim 3, wherein the normalizing microRNAs and the discriminating microRNAs are correlated from one or more of the following features:

| Features | Normalizers | Discriminator |
|---|---|---|
| A | hsa-let-7A | hsa-miR-146b |
| | hsa-miR-103 | |
| | hsa-miR-125a-5p | |
| B | hsa-let-7A | hsa-miR-152 |
| | hsa-miR-103 | |
| | hsa-miR-125a-5p | |
| C | hsa-let-7A | hsa-miR-155 |
| | hsa-miR-103 | |
| | hsa-miR-125a-5p | |
| D | hsa-let-7b | hsa-miR-200b |
| | hsa-miR-145 | |
| | RNU48 | |
| E | hsa-let-7b | hsa-miR-181b |
| | hsa-miR-145 | |
| | RNU48 | |
| F | hsa-miR-103 | hsa-miR-146b |
| | RNU48 | |
| G | hsa-miR-103 | hsa-miR-152 |
| | RNU48 | |
| H | hsa-miR-103 | hsa-miR-155 |
| | RNU48 | |
| I | hsa-miR-103 | hsa-miR-200b |
| | RNU48 | |
| J | hsa-miR-103 | hsa-miR-181b |
| | RNU48 | |
| K | hsa-miR-125a-5p | hsa-miR-146b |
| | hsa-let-7b | |
| L | hsa-miR-125a-5p | hsa-miR-152 |
| | hsa-let-7b | |
| M | hsa-miR-125a-5p | hsa-miR-155 |
| | hsa-let-7b | |
| N | hsa-miR-125a-5p | hsa-miR-200b |
| | hsa-let-7b | |
| O | hsa-miR-125a-5p | hsa-miR-181b |
| | hsa-let-7b | |
| P | hsa-let-7A | hsa-miR-146b |
| | hsa-miR-145 | |
| Q | hsa-let-7A | hsa-miR-152 |
| | hsa-miR-145 | |
| R | hsa-let-7A | hsa-miR-155 |
| | hsa-miR-145 | |
| S | hsa-let-7A | hsa-miR-200b |
| | hsa-miR-145 | |
| T | hsa-let-7A | hsa-miR-181b |
| | hsa-miR-145 | |

5. The method according to claim 1, wherein the thyroid nodule classification consists of at least one of: benign, malignant, medullary thyroid cancer, papillary thyroid carcinoma and its variants, follicular thyroid carcinoma and its variants, insular thyroid carcinoma, noninvasive follicular thyroid neoplasm with papillary-like nuclear features (NIFTP), goiter and its variants, thyroiditis and its variants, adenomas and their variants, Hurthle cells in thyroid and their variants, and thyroid hyperplasias and their variants.

6. The method according to claim 1, wherein when the classification is for medullary thyroid cancer, said discriminating microRNA is at least hsa-miR-375.

7. The method according to claim 1, further comprising the steps of:
a) collecting thyroid tissue sample;
b) extracting nucleic acids from the sample of step (a);
c) measuring the expression level of at least one normalizing microRNA and at least one discriminating microRNA; and
d) correlating the data obtained in step (c) of the expression level of at least one normalizing microRNA and at least one discriminating microRNA.

8. The method according to claim 7, wherein step (a) is performed by fine-needle aspiration puncture or solid or liquid biopsy; wherein step (c) is performed using a technique selected from the group consisting of RT-PCR, qPCR, sequencing, Nanostring, PCR-Digital, microarrays, fragment analysis, gel electrophoresis, immunohistochemistry, mass spectrometry, and combinations thereof; and wherein step (d) is performed by using an algorithm.

9. The method according to claim 7, further comprising the steps of:
a1) preparing the sample collected in step (a) before performing step (b);
b1) purifying the nucleic acids obtained in step (b);
b2) cDNA synthesis from the nucleic acids obtained in step (b1); and
b3) optionally, preamplification prior to step (c).

10. A kit for the classification of thyroid nodules, the kit comprising:
materials for measuring the expression level of at least one normalizing microRNA and at least one discriminating microRNA; and
at least one means for correlating the expression level of one to six normalizing microRNA with at least one discriminating microRNA;
wherein said normalizing microRNA is selected from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA, and combinations thereof, and
wherein said discriminating microRNA is selected from the group consisting of dme-miR-7, hsa-let-7a, hsa-let-7b, hsa-let-7e, hsa-let-7f, hsa-let-7g, hsa-miR-1, hsa-miR-101, hsa-miR-103, hsa-miR-106a, hsa-miR-106b, hsa-miR-10a, hsa-miR-1179, hsa-miR-122, hsa-miR-125a-3p, hsa-miR-125a-5p, hsa-miR-125b, hsa-miR-126, hsa-miR-130b, hsa-miR-133a, hsa-miR-136*, hsa-miR-136, hsa-miR-138, hsa-miR-144, hsa-miR-145, hsa-miR-146a, hsa-miR-146b, hsa-miR-149, hsa-miR-150, hsa-miR-151-5P, hsa-miR-152, hsa-miR-155, hsa-miR-15a, hsa-miR-16, hsa-miR-17, hsa-miR-181a, hsa-miR-181b, hsa-miR-183, hsa-miR-18a, hsa-miR-18b, hsa-miR-190, hsa-miR-191, hsa-miR-195, hsa-miR-197, hsa-miR-199a-3p, hsa-miR-199b, hsa-miR-200a, hsa-miR-200b, hsa-miR-200c, hsa-miR-203, hsa-miR-204, hsa-miR-205, hsa-miR-208, hsa-miR-208b, hsa-miR-20a, hsa-miR-20b, hsa-miR-21, hsa-miR-221, hsa-miR-222, hsa-miR-23b, hsa-miR-26a, hsa-miR-26b, hsa-miR-29a, hsa-miR-302c, hsa-miR-30a-3p, hsa-miR-30a-5p, hsa-miR-30c-2*, hsa-miR-30e-3p, hsa-miR-31, hsa-miR-3151, hsa-miR-346, hsa-miR-34a, hsa-miR-34c, hsa-miR-365, hsa-miR-375, hsa-miR-424, hsa-miR-425-5p, hsa-miR-449b, hsa-miR-503, hsa-miR-520b, hsa-miR-608, hsa-miR-613, hsa-miR-618, hsa-miR-642, hsa-miR-651, hsa-miR-7-2*, hsa-miR-885-5p, hsa-miR-9, hsa-miR-933, hsa-miR-99a, mmu-miR-137, mmu-miR-187, mmu-miR-451, RNU44, RNU48, U6 snRNA, and combinations thereof.

11. The kit according to claim 10, wherein said normalizing microRNA is selected from the group consisting of RNU48, hsa-miR-197, hsa-let-7b, hsa-miR-125a-5p, hsa-miR-103, hsa-let-7a, hsa-let-7e, and hsa-miR-145, and/or said discriminating microRNA is selected from the group consisting of hsa-miR-204, hsa-miR-152, hsa-miR-222, hsa-miR-181b, hsa-miR-146b, hsa-miR-155, hsa-miR-181a, hsa-miR-200b, hsa-miR-221, and combinations thereof.

12. The kit according to claim 10, further comprising:
material for collecting thyroid tissue sample or for extracting the existing thyroid material;
material for preparing said thyroid tissue sample;
reagents for extracting nucleic acids;
reagents for cDNA synthesis;
optionally, reagents for preamplification; and
material for measuring the expression level of microRNAs.

13. The method according to claim 5, wherein when the classification is for medullary thyroid cancer, said discriminating microRNA is at least hsa-miR-375.
